SEQUENCE LISTING (6 sequences)

SEQ ID NO: 1 - DNA, 24 bp, Artificial Sequence
  Description of Artificial SequenceFactor 5 wild type allele asymmetric PCR amplification upstream primer
tgaacccacagaaaatgatgcccn

SEQ ID NO: 2 - DNA, 26 bp, Artificial Sequence
  Description of Artificial SequenceFactor 5 wild type allele asymmetric PCR amplification downstream primer
ggaaatgccccattatttagccaggn

SEQ ID NO: 3 - DNA, 18 bp, Artificial Sequence
  Description of Artificial SequenceFactor 5 wild type allele asymmetric PCR amplification stabilized detection probe (blocker)
nnnnnngnnngnnnaggg

SEQ ID NO: 4 - DNA, 21 bp, Artificial Sequence
  Description of Artificial SequenceFactor 5 wild type allele asymmetric PCR amplification unstabilized detection probe (non-blocker)
ctgtattcctcgcctgtccag

SEQ ID NO: 5 - DNA, 18 bp, Artificial Sequence
  Description of Artificial SequenceFactor 5 wild type allele real-time PCR long unstabilized probe
caaggacaaaatacctgt

SEQ ID NO: 6 - DNA, 33 bp, Artificial Sequence
  Description of Artificial SequenceFactor 5 wild type allele real-time PCR long unstabilized probe
attcctcgcctgtccagggatctgctcttacag

(19) United States
(12) Patent Application Publication
(10) Pub. No.: US 2010/0143901 A1
(43) Pub. Date: Jun. 10, 2010

(54) Nuclease-Free Real-Time Detection of Nucleic Acids

(75) Inventor: Nicolas Newton, Oakland, CA (US)

Correspondence Address: Roche Molecular Systems, Inc.;Patent Law Department, 4300 Hacienda Drive, Pleasanton, CA, 94588, US

(73) Assignee: ROCHE MOLECULAR SYSTEMS, INC., Pleasanton, CA (US)

(21) Appl. No.: 12/330,694

(22) Filed: Dec. 9, 2008

(51) Int. Cl. C12Q 1/68

(52) U.S. Cl. 435/6

(57) ABSTRACT

The invention is a method for amplification and detection of nucleic acids using primers and at least one hybridization probe labeled with a first fluorescent moiety and a second moiety, capable of changing the fluorescence of said first fluorescent moiety. The method comprises the steps of effecting denaturation of said target, formation of hybrids between said primers and probe and said target and detecting the change in fluorescence of said first fluorescent moiety, upon formation of said hybrids. Reaction mixtures and kits for practicing the method of the present invention are also disclosed.

### FIELD OF THE INVENTION

The invention relates generally to the field of in vitro amplification and detection of nucleic acids. Specifically, it relates to the simultaneous amplification and detection of nucleic acids using fluorescently labeled probes, while the probes are not being hydrolyzed during amplification.

### BACKGROUND OF THE INVENTION

The polymerase chain reaction (PCR) has become a ubiquitous tool of biomedical research and diagnostics. Since the invention of PCR in the 1980s, there have been many modifications of the basic technology. One of the significant developments has been the advent of so called “real-time” assays, also called “homogeneous” assays, where the target nucleic acid is detected at the same time as it is being amplified by PCR. An advantage of such an assay is the ability to keep the sample vessel closed after the reaction is completed. The closed-tube protocol significantly reduces the risk of cross-contamination of samples as well as contamination of fresh samples by the existing amplification products. Moreover, real-time monitoring of amplification permits far more accurate quantification of starting target concentration as well as determining the efficiency of the amplification reaction.

A popular real-time assay takes advantage of a 5′-3′ nuclease activity of the DNA polymerase. This activity is employed to hydrolyze a sequence-specific labeled probe positioned downstream of one of the amplification primers. After hydrolysis the labeled oligonucleotide fragments are detected. In each cycle of amplification, the probe hybridizes to the target strand and is hydrolyzed by the 5′-3′ nuclease. The products of hydrolysis, labeled and unlabeled oligonucleotide fragments, accumulate in direct proportion to the accumulation of the amplification product.

A popular example of a nuclease probe is a fluorescently-labeled probe such as the TaqMan™ probe Typically, this type of probe is labeled by a pair of chromophores, forming a FRET (Foerster or Fluorescence Resonance Energy Transfer) pair. The two chromophores are either two fluorophores or a fluorophore and a non-fluorescent chromophore. The probe technology relies on the 5′-3′ nuclease activity of the DNA polymerase. Prior to the nuclease digestion, the chromophores of the probe interact in such a way that fluorescence of the desired wavelength is reduced. The nuclease digestion physically separates the chromophores, energy transfer no longer occurs, and emission of the desired wavelength increases above the background level.

Fortunately, many commercially used polymerases naturally possess the desired 5′-3′ nuclease activity. At the same time, many enzymes lacking this activity have also been isolated or developed. These nuclease-deficient enzymes have many superior properties, such as improved processivity, thermal stability and affinity to various non-traditional nucleotide substrates. However, to this date, one could not take advantage of these superior nuclease-free enzymes in a real-time assay, such as a TaqMan™ assay, since the nuclease activity was thought to be an essential part of the assay.

It is noted that certain FRET probes that do not rely on 5′-3′ nuclease are claimed in the art. For example, molecular beacon probes are described in Tyagi et al., (1996) Nature Biotechnology, 14:303-308. It is asserted that instead of nuclease activity, these probes employ the unfolding of a secondary structure as a way to separate the chromophores within the FRET pair. Molecular beacons incorporate an elaborate secondary structure that creates a close proximity between the chromophores and allows quenching to take place. When the probe hybridizes to the target sequence, the secondary structure unravels, separating the FRET pair and allowing the desired fluorescence to occur.

Another example of a hybridization probe that does not require a nuclease is an MGB Eclipse™ probe described in U.S. Pat. No. 5,801,155 and its continuations. These probes have been developed for allele discrimination in a probe melting assay. An MGB Eclipse™ probe is an oligonucleotide with a 5′-end capped by a molecule derived from a naturally occurring antibiotic. The 5′-terminal cap promotes the minor groove binding (MGB) property of the probe. As an extra benefit, it is noted that the 5′-terminal cap makes the probe resistant to nuclease digestion.

Although it is asserted that molecular beacons and MGB Eclipse™ probes do not require the 5′-3′ nuclease activity, they have their own drawbacks, such as cost and complexity. With respect to molecular beacons, the target sequence does not always allow for the formation of the stem-loop secondary structure, requiring that additional sequences be incorporated into the probe. MGB Eclipse™ probes include a proprietary 5′-terminal cap. By comparison, simple hybridization probes, such as TaqMan™ probes, are freely available, versatile and less costly.

### SUMMARY OF THE INVENTION

The present invention comprises a method for amplification and detection of a target nucleic acid in a sample comprising the steps of: (a) contacting a sample, possibly comprising a target nucleic acid, with a template-dependent nucleic acid polymerase, substantially lacking 5′-3′ nuclease activity, at least two primers, at least partially complementary to separate portions of said target, and at least one probe, at least partially complementary to a portion of said target, other than the portions complementary to said primers; wherein said probe has a first fluorescent moiety and a second moiety, capable of changing the fluorescence of said first fluorescent moiety; (b) subjecting the mixture of step (a) to conditions sufficient to permit denaturation of said target; (c) subjecting the mixture of step (b) to conditions sufficient to permit said primers and probe to form hybrids with said target; and (d) detecting the change in fluorescence of said first fluorescent moiety, upon formation of said hybrids. Optionally, the invention comprises repeating steps (b)-(d) multiple times. Reaction mixtures and kits for practicing the invention are also disclosed.

### BRIEF DESCRIPTION OF THE DRAWINGS

FIG. 1 shows amplification and detection of various amounts of target with nuclease-proficient and nuclease-deficient DNA polymerase, according to Example 1.

### DETAILED DESCRIPTION OF THE INVENTION

Definitions

The following definitions apply to the terms used throughout the application.

A “5′-3′ nuclease activity” or “5′ to 3′ nuclease activity” is the activity of a DNA polymerase that cleaves the 5′ terminal nucleotide or nucleotides of at least one strand in a double-stranded DNA. In one example of the 5′-3′ nuclease activity, the 5′-3′ nuclease activity of the Taq polymerase releases mono- and oligonucleotides from the 5′ end of a hybridized strand located downstream of the primer being extended by the same polymerase.

The terms “nucleic acid polymerase substantially lacking the 5′-3′ nuclease activity” or “5′-3′-nuclease-deficient enzyme”, or for simplicity, “nuclease-deficient enzyme” refer to a polymerase that has 50% or less of the 5′-3′ activity than Taq DNA polymerase. The methods of measuring the 5′-3′ nuclease activity and conditions for measurement have been described in U.S. Pat. No. 5,466,591. The examples of polymerases lacking the 5′-3′ nuclease activity include the Stoffel fragment of Taq DNA polymerase (U.S. Pat. No. 5,466,591), mutants of Thermus africanus DNA polymerase (U.S. Pat. No. 5,968,799), mutants of Thermotoga maritima DNA polymerase (U.S. Pat. Nos. 5,624,833 and 5,420,029), mutants of Thermus species sps17 and Thermus species Z05 DNA polymerases (U.S. Pat. Nos. 5,466,591 and 5,405,774). 5′-3′ nuclease enzymes may also be chimeras, i.e. chimeric proteins, composed of domains derived from m species and having mutations that eliminate the 5′-3′ nuclease activity (U.S. Pat. Nos. 5,795,762 and 6,228,628).

An “asymmetric PCR” is a PCR wherein the initial amounts of two amplification primers are unequal. The primers are referred to as “excess primer” and “limiting primer.” The strand resulting from extension of the excess primer is accumulated in excess and is called “the excess strand.” The other strand, resulting from extension of the limiting primer, is accumulated in smaller amounts and is called “the limiting strand.”

“FRET” or “fluorescent resonance energy transfer” or “Foerster resonance energy transfer” is a transfer of energy between at least two chromophores, a donor chromophore and an acceptor chromophore (referred to as a quencher). The donor typically transfers the energy to the acceptor when the donor is excited by light radiation with a suitable wavelength. The acceptor typically re-emits the transferred energy in the form of light radiation with a different wavelength. When the acceptor is a “dark” quencher, it dissipates the transferred energy in a form other than light. Whether a particular fluorophore acts as a donor or an acceptor depends on the properties of the other member of the FRET pair. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Calif.), Iowa Black™, (Integrated DNA Tech., Inc., Coralville, Iowa), BlackBerry™ Quencher 650 (BBQ-650), (Berry & Assoc., Dexter, Mich.).

A “chromophore” is a compound or a moiety attachable to a biomolecule, for example, a nucleic acid, which is capable of selective light absorption resulting in coloration. A chromophore may or may not emit light radiation when excited.

A “fluorescent dye” or a “fluorophore” is a fluorescent chromophore. A fluorophore is capable of emitting light radiation when excited by a light of a suitable wavelength. Examples of fluorescent dyes include rhodamine dyes, cyanine dyes, fluorescein dyes and BODIPY® dyes.

A “hybridization” is an interaction between two usually single-stranded or at least partially single-stranded nucleic acids, Hybridization occurs as a result of base-pairing between nucleobases and involves physicochemical processes such as hydrogen bonding, solvent exclusion, base stacking and the like. Hybridization can occur between fully-complementary or partially complementary nucleic acid strands. The ability of nucleic acids to hybridize is influenced by temperature and other hybridization conditions, which can be manipulated in order for the hybridization of even partially complementary nucleic acids to occur. Hybridization of nucleic acids is well known in the art and has been extensively described in Ausubel (Eds.) Current Protocols in Molecular Biology, v. I, II and III (1997).

A “label” refers to a moiety attached (covalently or non-covalently), to a molecule, which moiety is capable of providing information about the molecule. Exemplary labels include fluorescent labels, radioactive labels, and mass-modifying groups.

A “modified enzyme” refers to an enzyme comprising a protein in which at least one amino acid differs from the corresponding amino acid in a reference sequence of amino acids (native or wild-type sequence). Exemplary modifications include insertions, deletions, and substitutions of one or more amino acids. Modified enzymes also include chimeric enzymes that have identifiable component sequences derived from two or more parent enzymes.

A “modified nucleotide” refers to a nucleotide that includes one or more non-naturally occurring moieties. In some embodiments, the modified nucleotides include non-naturally occurring bases or sugar moieties, including bases and sugar moieties substituted with additional chemical groups. Some examples of modified nucleotides can be found in U.S. Pat. No. 6,001,611. Typically, modified nucleotides can be incorporated into a nucleic acid and modify certain properties of the nucleic acid. For example, modified nucleotides can alter melting temperature and ability to be extended by a nucleic acid polymerase, especially in the presence of a mismatch.

A “nucleic acid” refers to polymers of nucleotides (e.g., ribonucleotides and deoxyribonucleotides, both natural and non-natural) such polymers being DNA, RNA, and their subcategories, such as cDNA, mRNA, etc. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanosine, cytosine, uracil and thymidine) as well as non-natural bases. The example of non-natural bases include those described in, e.g., Seela et al. (1999) Helv. Chim. Acta 82:1640. Certain bases used in nucleotide analogs act as melting temperature (Tₘ) modifiers. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, which is incorporated herein by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytidine; 5-fluorocytidine; 5-chlorocytidine; 5-iodocytidine; 5-bromocytidine; 5-methylcytidine; 5-propynylcytidine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

A “nucleic acid polymerase” or simply “polymerase” refers to an enzyme that catalyzes the incorporation of nucleotides into a nucleic acid.

An “oligonucleotide” refers to a short nucleic acid, typically ten or more nucleotides in length. Oligonucleotides are prepared by any suitable method known in the art, for example, direct chemical synthesis as described in Narang et al. (1979) Meth. Enzymol. 68:90-99; Brown et al. (1979) Meth. Enzymol. 68:109-151; Beaucage et al. (1981) Tetrahedron Lett. 22:1859-1862; Matteucci et al. (1981) J. Am. Chem. Soc. 103:3185-3191; or any other method known in the art.

A “primer” is an oligonucleotide, which is capable of acting as a point of initiation of extension along a complementary strand of a template nucleic acid. A primer that is at least partially complementary to a subsequence of a template nucleic acid is typically sufficient to hybridize with template nucleic acid and for extension to occur.

A “primer extension” refers to a chemical reaction where one or more nucleotides have been added to the primer.

A “probe” refers to a labeled oligonucleotide which forms a duplex structure with a sequence in the target sequence, due to at least partial complementarity of the probe and the target sequence.

A “template” or “target” refers to a nucleic acid which is to be amplified, detected or both. The target or template is a sequence to which a primer or a probe can hybridize. Template nucleic acids can be derived from essentially any source, including microorganisms, complex biological mixtures, tissues, bodily fluids, sera, preserved biological samples, environmental isolates, in vitro preparations or the like. The template or target may constitute all or a portion of a nucleic acid molecule.

A “thermostable nucleic acid polymerase” or “thermostable polymerase” is a polymerase enzyme, which is relatively stable at elevated temperatures when compared, for example, to polymerases from E. coli. As used herein, a thermostable polymerase is suitable for use under temperature cycling conditions typical of the polymerase chain reaction (“PCR”).

It has been discovered that the traditional real-time PCR assay may be performed without the nuclease digestion of the probe. Specifically, it has been discovered that simple oligonucleotide hybridization probes, lacking any complex chemical modifier groups or specially designed secondary structure, can be used to detect amplification of nucleic acids without the 5′-3′ nuclease cleavage of the probe. The inventors have shown that even in the absence of nuclease cleavage, the probes generate a detectable change in fluorescent signal upon binding to the target and this signal increases in proportion to the accumulation of the amplicon. The continuous detection of the signal is sufficient to generate data sets comparable to those of traditional nuclease-based real-time assays.

Amplification of nucleic acid sequences, both RNA and DNA, is described in U.S. Pat. Nos. 4,683,195, 4,683,202, and 4,965,188. The preferred method, polymerase chain reaction (PCR), typically is carried out using a thermostable DNA polymerase, which is able to withstand the temperatures used to denature the amplified product in each cycle. PCR is now well known in the art and has been described extensively in the scientific literature. See, for example, PCR Applications, ((1999) Innis et al., eds., Academic Press, San Diego), PCR Strategies, ((1995) Innis et al., eds., Academic Press, San Diego); PCR Protocols, ((1990) Innis et al., eds., Academic Press, San Diego), and PCR Technology, ((1989) Erlich, ed., Stockton Press, New York), each incorporated herein by reference. A review of amplification methods is provided in Abramson and Myers, ((1993) Current Opinion in Biotechnology 4:41-47), incorporated herein by reference.

A traditional real-time PCR amplification and detection using the 5′-3′ nuclease (“hydrolysis assay”) is described in Holland et al., (1991) Proc. Natl. Acad. Sci. 88:7276-7280 and U.S. Pat. No. 5,210,015. The basic protocol involves (1) contacting a sample comprising single-stranded nucleic acid targets with a least one extendible oligonucleotide primer and at least one labeled oligonucleotide probe, located downstream of the primer under the conditions, wherein the probe and the primer form hybrids with their respective complementary sequences; (2) maintaining the sample with a nucleic acid polymerase having a 5′-3′ nuclease activity, so that said activity cleaves the annealed probe and releases the labeled fragments; and (3) detecting and measuring the release of labeled fragments.

The present invention enables the use of 5′-3′ nuclease-deficient polymerases with the FRET-type probe. For example, the invention enables the use of 5′-3′ nuclease-deficient thermostable polymerases in a real-time amplification set-up. 5′-3′ nuclease deficient polymerases are exemplified by the Klenow fragment of E. coli DNA Polymerase I. 5′-3′ nuclease-deficient thermostable polymerases have been isolated from several species: for example, Thermus Stoffel fragment (U.S. Pat. No. 5,466,591), Thermotoga (U.S. Pat. Nos. 5,420,029, 5,466,591 and 5,948,614 and other species. These 5′-3′-nuclease-deficient polymerases have been shown to have several superior properties as compared the nuclease-proficient enzymes. The superior properties include increased thermal stability (see U.S. Pat. Nos. 5,466,591 and 5,948,614), increased processivity (see U.S. Pat. No. 5,466,591), reduced pyrophosphorolysis, increased tolerance for certain modified nucleotides (see U.S. Pat. No. 6,228,628) as well as higher PCR product yields (see U.S. Pat. No. 5,466,591).

Despite the many advantages of the 5′-3′ nuclease-deficient enzymes, prior to the present invention, these enzymes were incompatible with traditional real-time PCR applications. It was believed that to generate an amplification-dependent signal, a fluorescent hybridization probe must be hydrolyzed into fragments (see U.S. Pat. No. 5,210,015). Specifically it was believed that the donor and the acceptor fluorophores had to be separated by hydrolysis (see U.S. Pat. No. 5,538,848). The present invention demonstrates that hydrolysis by a nuclease is not necessary and that a 5′-3′-deficient enzyme can be successfully used to generate the amplification-dependent signal.

Obviating the need for the 5′-3′-nuclease digestion provides certain advantages to the assay. Specifically, in later cycles of PCR, the strands of the nascent amplicon effectively compete with the probe in hybridizing with each other. Because the amplicon strands are longer than the probe, the thermodynamics and kinetics favor the amplicon duplex formation and disfavor the binding of the probe. This problem is exacerbated by hydrolysis of the probe by the nuclease during amplification. Since the probe hydrolysis was thought to be an indispensable part of a traditional nuclease assay, one had no choice but to supply large amounts of probe to ensure that enough is available in later cycles of PCR. The present invention overcomes the problem by eliminating probe hydrolysis.

The present invention employs a probe labeled with two interacting chromophores. The chromophores can be two fluorophores or a fluorophore and a non-fluorescent (“dark”) quencher. An example of this type of probe is described in U.S. Pat. No. 5,210,015. These probes employ fluorophore quenching resulting from the Foerster Resonance Energy Transfer (FRET) phenomenon. When two chromophores form a FRET pair, each chromophore's emission is affected by the transfer of the energy to or from the other chromophore. Livak et al. ((1995) PCR Methods Appl., 4:357-362) provide a detailed study of how the interaction between chromophores changes depending on the distance between them on a hybridized and non-hybridized nucleic acid strands.

In the present invention, the probe is labeled with a pair of interacting chromophores, at least one of which is a fluorescent signal-generating label, positioned so that the detectable signal is at least partially quenched when the probe is in the unhybridized form. The detectable signal (or an increase in the detectable signal) is generated when the probe hybridizes to the target sequence. In the prior art real-time PCR methods (such as e.g. the method described in the U.S. Pat. No. 5,478,972); the detectable signal is generated when the hybridized probe is hydrolyzed by the 5′-3′ nuclease activity of the DNA polymerase. In the present invention, detectable signal (or increase in the detectable signal) is generated when the probe hybridizes to the target sequence. Thus the probe is not consumed by the nuclease in the course of each amplification cycle.

Typical examples of fluorescent dyes are rhodamine dyes (R6G, R110, TAMRA, ROX, etc.), cyanine dyes (Cy3, Cy3.5, Cy5, Cy5.5, etc.), fluorescein dyes (JOE, VIC, TET, HEX, FAM, etc.), BODIPY® dyes (FL, 530/550, TR, TMR, etc.), ALEXA FLUOR® dyes (488, 532, 546, 568, 594, 555, 653, 647, 660, 680, etc.) and dichlororhodamine dyes and the like. Examples of non-fluorescent quenchers are Black Hole Quenchers™ (BHQ), Iowa Black™ and BlackBerry™ Quencher 650-dt (BBQ-650-dt).

In the context of the present invention, generally the 3′ terminus of the probe will be “blocked” to prohibit extension of the probe by the DNA polymerase. “Blocking” may be achieved by any method known in the art, such as using non-complementary bases or by adding a chemical moiety such as a phosphate group, biotin or a dye to the 3′ or 2′ position of the sugar moiety of the last nucleotide.

The present invention provides a simplification of the traditional real-time amplification assay that allows the option to use less probe without sacrificing sensitivity of the assay. Additionally, the invention opens the door to the use of superior 5′-3′ nuclease-free polymerases in the real-time amplification assay.

One aspect of the present invention provides a method of simultaneous amplification and detection of nucleic acids using labeled probes. The method includes incubating the template nucleic acid with at least one primer and at least one labeled probe (both primer and probe being at least partially complementary to separate portions of the template sequence), and a polymerase substantially free of the 5′-3′ nuclease activity, under the conditions suitable for the extension of the primer or primers by the polymerase. These conditions include the presence of a suitable buffer, nucleoside triphosphates and a temperature profile permitting template denaturation, primer annealing, primer extension by the polymerase and probe annealing.

In another aspect, the invention includes providing conditions suitable for repeated cycles of amplification, such as by polymerase chain reaction (PCR). These conditions include a temperature profile permitting repeated cycles of template denaturation, primer annealing, probe annealing and primer extension by the polymerase.

In yet another aspect, the invention includes simultaneous amplification and detection of the target nucleic acid and its amplicon. The detection of the fluorescent signal (or increase in the fluorescent signal) is indicative of the presence or accumulation of the target nucleic acid and its amplicon. The methods and devices for detecting fluorescence are well known in the art. One of the suitable methods involves the use of thermocyclers with an optical module, such as the LightCycler™ family of instruments or its equivalents.

In yet another aspect, the reaction conditions include asymmetric PCR, wherein the excess strand is the strand complementary to the probe. Without being bound by a particular theory, the inventors suggest that asymmetric PCR may be beneficial in later cycles of amplification, where the concentration of nascent amplicon strands increases and creates unfavorable kinetic conditions for probe binding. This effect may be minimized if the strand complementary to the probe is present in excess.

In yet another aspect of the invention, real-time amplification and detection using 5′-3′ nuclease deficient enzymes may be combined with other methods that require the presence of the 5′-3′ nuclease deficient enzyme. For example, a method of rare mutation detection that relies on a 5′-3′ nuclease deficient enzyme has been described in U.S. Pat. No. 5,849,497 and application Ser. No. 12/186,311, filed on Aug. 5, 2008. The method involves blocking amplification of a wild-type sequence with an oligonucleotide that specifically binds to the wild-type but not the mutant sequence. The suppression of amplification is only possible if the amplification enzyme lacks the 5′-3′ nuclease activity.

In another aspect of the invention, the detection method involves a probe melting assay, where the amplification products are detected and identified by determining their unique melting temperatures (Tₘ). A melting assay measures a change in a detectable parameter (such as fluorescence) associated with the change in temperature. The increase in temperature that results in melting of the template-probe hybrid is accompanied by a measurable change in fluorescence. Measuring the temperature-dependent change in fluorescence of a dye or dyes conjugated to a pair of probes or to a single probe has been described in the U.S. Pat. No. 6,174,670. Identification of a particular genotype by its unique Tₘ with a pair of labeled probes has been described in De Silva et al., (1998) “Rapid genotyping and quantification on the LightCycler™ with hybridization probes,” Biochemica, 2:12-15. The method of the present invention is particularly suitable for being combined with the melting assay because the detection probe is not consumed by the 5′-3′ nuclease during amplification. Therefore a sufficient amount of the probe is available for post-amplification melting assay.

In yet another aspect, the invention provides a reaction mixture comprising at least one hybridization probe labeled with two interacting fluorophores according to the invention, at least one primer, a nucleic acid polymerase substantially free of the 5′-3′ nuclease activity, and other reagents necessary for the amplification of nucleic acids, including nucleoside triphosphates and organic and inorganic ions; as well as optional reagents, such as uracil-N-DNA glycosylase (UNG) for prevention of carryover contamination and pyrophosphatase for prevention of pyrophosphorolysis.

In yet another aspect, the invention provides a kit for the amplification and detection of nucleic acids. The kit includes (a) a nucleic acid polymerase sufficiently free of 5′-3′ nuclease activity; (b) at least one probe labeled with two interacting fluorophores; (c) at least one primer; (d) a solution of organic and inorganic ions; and (e) nucleoside triphosphates. Optionally, the kit also includes an amount of template nucleic acid. As a further option, the kit may include one or more of the following: uracil-N-DNA glycosylase (UNG) for prevention of carryover contamination and pyrophosphatase for prevention of pyrophosphorolysis.

In some embodiments, the primer nucleic acid is attached to a solid support. In some embodiments, the primer comprises a label, such as a radioisotope, a fluorescent dye, other than the fluorescent dyes attached to the probe, a mass-modifying group, or the like.

### EXAMPLE I

Amplification and Detection of Various Amounts of Target with the Nuclease-Deficient and Nuclease-Proficientpolymerase

In this example, the method of the present invention was used to amplify a region of the human Factor V gene that includes the site of the Leiden mutation, cloned into a plasmid vector. The asymmetric PCR was conducted with a seven-fold excess of the excess primer over the limiting primer. The detection was performed with a hybridization probe labeled with a fluorescein dye and a BlackHole™ quencher as shown in Table 1. The probe was designed to hybridize to the excess strand.

TABLE 1
Primers and probes

| | | |
|---|---|---|
| Upstream primer | SEQ ID NO.: 1 | 5′-TGAACCCACAGAAAATGATGCCCE-3′ |
| Downstream primer | SEQ ID NO.: 2 | 5′-GGAAATGCCCCATTATTTAGCCAGGE-3′ |
| Probe | SEQ ID NO.: 4 | 5′-FCTGTATTCCTCGCCTGTCCAGQp-3′ |

E = para-t-butyl benzyl dA
F = cx-FAM
Q = BHQ2
p = 3′-phosphate

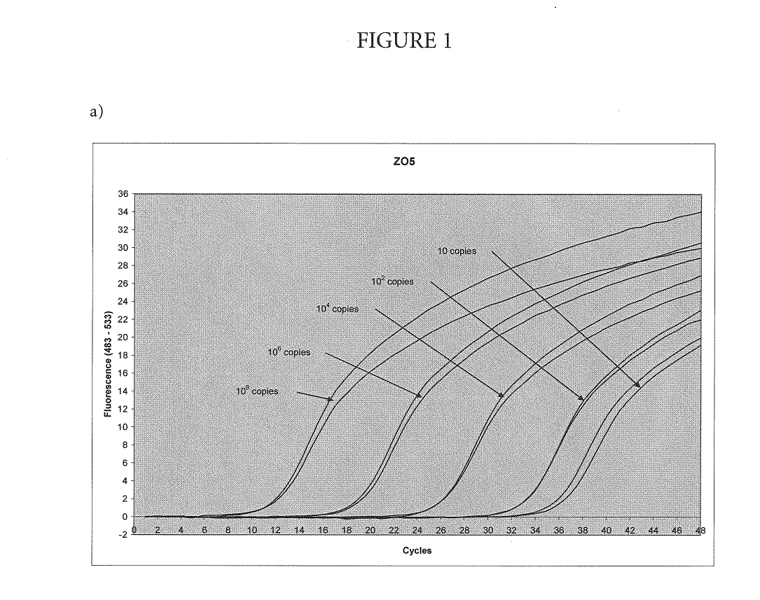

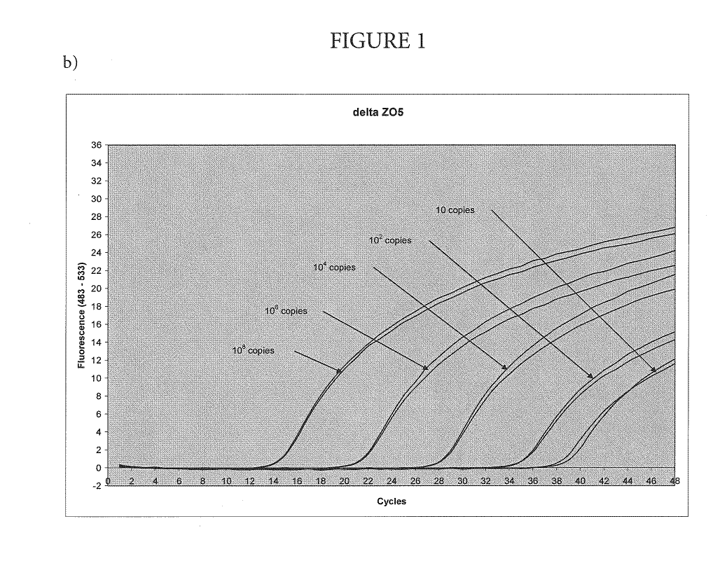

Each 100 μL reaction contained an amount of target DNA (between 10 and 10⁸ copies, as indicated on FIG. 1) 5% glycerol; 50 mM Tricine, pH 8.3; 25 mM potassium acetate; 200 μM of each dATP, dGTP and dCTP, 400 μM dUTP; 0.7 μM upstream (excess) primer (SEQ ID NO.: 1); 0.1 μM downstream (limiting) primer (SEQ ID NO: 2); 0.4 μM probe (SEQ ID NO: 3); 0.04 U/μL uracil-N-glycosylase (UNG); 0.4 U/μL ZO5 or ΔZO5 DNA polymerase; and 4 mM magnesium acetate.

The amplification and detection were performed using the Roche LightCycler™ LC480 instrument. The reactions were subjected to the following temperature profile: 50° C. for 5 minutes (UNG step); 2 cycles of 94° C. for 15 seconds and 59° C. for 40 seconds, followed by 48 cycles of 91° C. for 15 seconds and 59° C. for 40 seconds. The fluorescence data were collected during each 59° C. step.

The results are shown in FIG. 1. The data is expressed as fluorescence units in the 483-533 nm filter channel, plotted against the number of amplification cycles. The initial number of copies of the target DNA is indicated for each curve. Panel “a” represents data generated with a polymerase possessing the 5′-3′ nuclease activity. Panel “b” represents data generated with a polymerase deficient in the 5′-3′ nuclease activity. The data shows a steady increase in the fluorescent signal in each reaction.

While the invention has been described in detail with reference to specific examples, it will be apparent to one skilled in the art that various modifications can be made within the scope of this invention. Thus the scope of the invention should not be limited by the examples described herein, but by the claims presented below.

1. A method for amplification and detection of a target nucleic acid in a sample comprising the steps of:
(a) contacting a sample, possibly comprising a target nucleic acid, with a template-dependent nucleic acid polymerase, substantially lacking 5′-3′ nuclease activity, at least two primers, at least partially complementary to separate portions of said target, and at least one probe, at least partially complementary to a portion of said target, other than the portions complementary to said primers; wherein
said probe has a first fluorescent moiety and a second moiety, capable of changing the fluorescence of said first fluorescent moiety;
(b) subjecting the mixture of step (a) to conditions sufficient to permit denaturation of said target;
(c) subjecting the mixture of step (b) to conditions sufficient to permit said primers and probe to form hybrids with said target; and
(d) detecting the change in fluorescence of said first fluorescent moiety, upon formation of said hybrids.

2. The method of claim 1, wherein the sequence of steps (b)-(d) is repeated multiple times.

3. The method of claim 1, wherein said at least two primers are two primers present in unequal amounts.

4. The method of claim 1, wherein said first fluorescent moiety is selected from a group consisting of rhodamine dyes, cyanine dyes, fluorescein dyes and BODIPY® dyes, ALEXA FLUOR® dyes and dichlororhodamine dyes.

5. The method of claim 1, wherein said second moiety is selected from a group consisting of TAMRA, Black Hole Quenchers, DABCYL, Iowa Black and BlackBerry Quencher 650.

6. The method of claim 1, wherein said nucleic acid polymerase substantially lacking the 5′-3′ nuclease activity is derived from one or more species selected from a group consisting of Thermus aquaticus, Thermus species sps17, Thermus species Z05, Thermotoga maritima and Thermus africanus.

7. A reaction mixture for amplification and detection of a target nucleic acid, comprising template-dependent nucleic acid polymerase, substantially lacking the 5′-3′ nuclease activity, at least two primers, at least partially complementary to separate portions of said target, and at least one probe oligonucleotide, at least partially complementary to a portion of said target, other than the portions complementary to said primers; wherein said probe as a first fluorescent moiety and a second moiety, capable of changing the fluorescence of said first fluorescent moiety.

8. The reaction mixture of claim 7, further comprising an amount of target nucleic acid.

9. The reaction mixture of claim 7, further comprising a reagent useful for prevention of carryover contamination.

10. The reaction mixture of claim 7, further comprising a reagent useful for prevention of pyrophosphorolysis.

11. The reaction mixture of claim 7, wherein said first fluorescent moiety is selected from a group consisting of rhodamine dyes, cyanine dyes, fluorescein dyes and BODIPY® dyes, ALEXA FLUOR® dyes and dichlororhodamine dyes.

12. The reaction mixture of claim 7, wherein said second moiety is selected from a group consisting of TAMRA, Black Hole Quenchers, DABCYL, Iowa Black and BlackBerry Quencher 650.

13. The reaction mixture of claim 7, wherein said nucleic acid polymerase substantially lacking the 5′-3′ nuclease activity is derived from one or more species selected from a group consisting of Thermus aquaticus, Thermus species sps17, Thermus species Z05, Thermotoga maritima and Thermus africanus.

14. The reaction mixture of claim 7, wherein said at least two primers are two primers present in unequal amounts.

15. A kit for amplification and detection of a target nucleic acid, comprising: template-dependent nucleic acid polymerase, substantially lacking 5′-3′ nuclease activity;
at least two primers, at least partially complementary to separate portions of said target; and
at least one probe, at least partially complementary to a portion of said target, other than the portions complementary to said primers; wherein
said probe has a first fluorescent moiety and a second moiety, capable of changing the fluorescence of said first fluorescent moiety.

16. The kit of claim 15, further comprising an amount of target nucleic acid.

17. The kit of claim 15, further comprising a reagent useful for prevention of carryover contamination.

18. The kit of claim 15, further comprising a reagent useful for prevention of pyrophosphorolysis.

19. The kit of claim 15, wherein said first fluorescent moiety is selected from a group consisting of rhodamine dyes, cyanine dyes, fluorescein dyes and BODIPY® dyes, ALEXA FLUOR® dyes and dichlororhodamine dyes.

20. The kit of claim 15, wherein said second moiety is selected from a group consisting of TAMRA, Black Hole Quenchers, DABCYL, Iowa Black and BlackBerry Quencher 650.

21. The kit of claim 15, wherein said at least two primers are two primers present in unequal amounts.

22. The kit of claim 15, wherein said nucleic acid polymerase substantially lacking the 5′-3′ nuclease activity is derived from one or more species selected from a group consisting of Thermus aquaticus, Thermus species sps17, Thermus species Z05, Thermotoga maritima and Thermus africanus.
